(19) United States
(12) Patent Application Publication
(10) Pub. No.: US 2008/0053454 A1
(43) Pub. Date: Mar. 6, 2008

(54) Endotracheal tube including a partially inverted cuff collar

(75) Inventors: Pedro Velazco Pasillas, Juarez (MX); Joel Colburn, Walnut Creek, CA (US); Michael Vannier, Danville, CA (US)

Correspondence Address: BAKER BOTTS L.L.P.;PATENT DEPARTMENT, 98 SAN JACINTO BLVD., SUITE 1500, AUSTIN, TX, 78701-4039, US

(73) Assignee: Nellcor Puritan Bennett Incorporated

(21) Appl. No.: 11/469,624

(22) Filed: Sep. 1, 2006

(51) Int. Cl. A62B 9/06; A61M 16/00

(52) U.S. Cl. 128/207.15; 128/207.14

(57) ABSTRACT

A medical device may include a tubular body configured to communicate gas, and an inflatable cuff coupled to the tubular body at least by a collar. The collar may include a fold such that a first portion of the collar overlies a second portion of the collar in a concentric manner.

### TECHNICAL FIELD

The present disclosure relates generally to the field of medical devices, e.g., a medical tube (e.g., an endotracheal tube) including an inflatable cuff having a partially inverted collar.

### BACKGROUND

Conventional methods of endotracheal intubation involve the insertion of a tubular device, e.g., an endotracheal tube, into the trachea. The endotracheal tube typically passes through the trachea and terminates above the carina, allowing gases to be directed through the tube and into the lungs.

A primary objective of this type of treatment is the mechanical ventilation of a subject's lungs, which may be required or appropriate due to the subject's medical condition. In order to create the air pressure necessary to artificially ventilate the lungs, the passageways around the tube are typically sealed, which may be accomplished, e.g., using an inflatable cuff provided around the tube. With the tube in place, the cuff is typically located within the trachea about 3-5 centimeters above the carina. The cuff may then be inflated to expand and seal against the wall of the trachea, thereby preventing gases being pumped into the lungs from backing up around the tube.

Although this method of treatment has been relatively successful, problems remain. For example, with cuffed endotracheal tubes, secretions may collect proximate the cuff, providing a site for the possible accumulation of pathogens. Various methods have been devised for removing such secretions. For example, a small opening may be provided above the cuff with an associated suction lumen. Fluids and/or solids (e.g., secretions) can be periodically or continuously removed through the opening and lumen by suction.

Cuffed endotracheal tubes often do not self-center within the trachea upon inflation of the cuff, and as a result, the suction openings of a particular tube may not be spaced apart from the tracheal wall. For example, due to the curvature of the tube and/or other factors, the suction opening may be located very near the tracheal wall upon cuff inflation. In some instances, the suction opening may actually contact the tracheal wall. In such situations, the tracheal wall membrane may be drawn into the suction opening upon application of a vacuum, thereby occluding the opening. This may prevent the proper removal of secretions from the subglottic space and/or may cause trauma to the tracheal wall.

### SUMMARY

In one embodiment of the present disclosure, a medical device may include a tubular body configured to communicate gas, and an inflatable cuff coupled to the tubular body at least by a collar. The collar may include a fold such that a first portion of the collar overlies a second portion of the collar in a concentric manner.

In another embodiment of the present disclosure, a medical device may include conveying means for channeling gas to an area of the body, securing means for securing the conveying means against the wall of a body cavity, and attaching means for attaching the securing means to the conveying means. The attaching means may include a fold such that a first portion of the attaching means overlies a second portion of the attaching means in a concentric manner.

In another embodiment of the present disclosure, a method for intubation may be provided. A tube including an inflatable cuff coupled to an tubular body by a collar may be inserted into a body cavity of a subject. The collar may include a fold such that a first portion of the collar overlies a second portion of the collar in a concentric manner. The cuff may be inflated within the body cavity, and gas may be conveyed to an area of the body through the tubular body.

In another embodiment of the present disclosure, a method of attaching an inflatable cuff having a collar to a tubular body is provided. The method may include mounting the inflatable cuff on the tubular body and manipulating the inflatable cuff to configure the collar such that a first portion of the collar overlies a second portion of the collar in a concentric manner.

### BRIEF DESCRIPTION OF THE DRAWINGS

FIG. 1 illustrates a medical tube including an inflatable cuff having a partially inverted collar, in accordance with an example embodiment of the disclosure;

FIG. 2 is an enlarged view of the inflatable cuff shown in FIG. 1, in accordance with one embodiment of the disclosure;

FIGS. 3A-3G illustrate an example method of configuring and attaching the inflatable cuff shown in FIG. 2 to a tubular body of a medical tube, in accordance with one embodiment of the disclosure;

FIG. 4 illustrates an example embodiment in which a band is positioned around a collar of an inflatable cuff, according to one embodiment of the disclosure;

FIG. 5 illustrates an example inflatable cuff including a collar spacer, in accordance with one embodiment of the disclosure; and

FIG. 6 is a flow diagram of a method of intubation of a tube having an inflatable cuff with a partially inverted collar, in accordance with one embodiment of the disclosure.

### DETAILED DESCRIPTION OF THE DRAWING

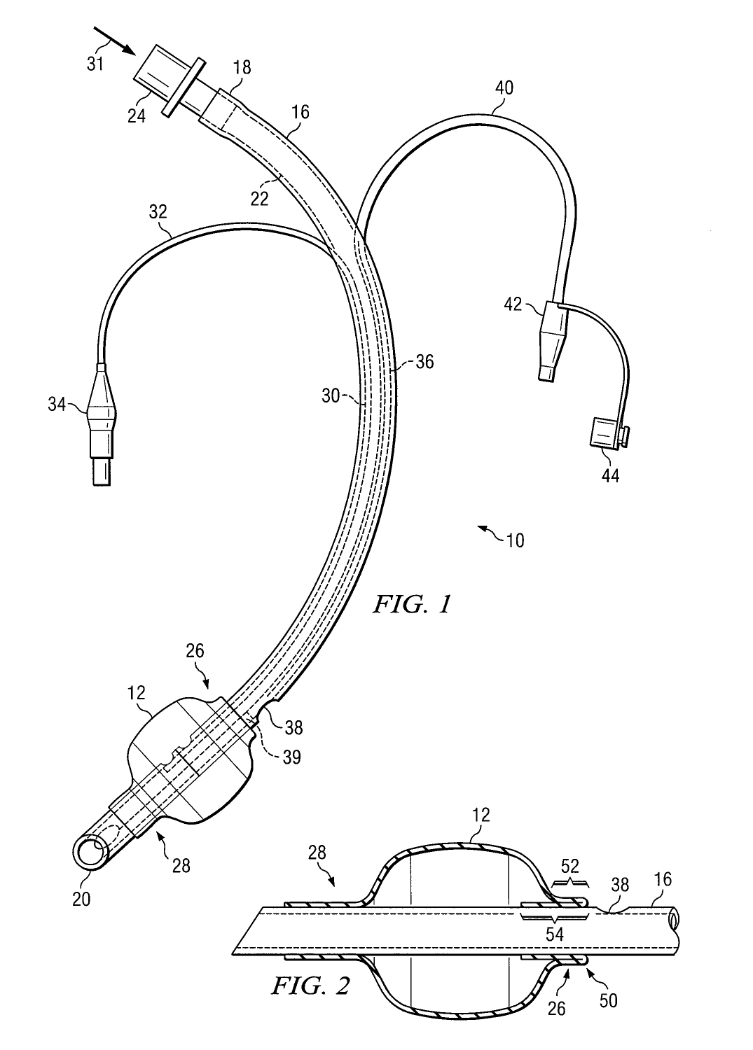

Selected embodiments of the disclosure may be understood by reference, in part, to FIGS. 1-6, wherein like numbers refer to same and like parts. The present disclosure is broadly concerned with medical tubes (e.g., endotracheal, tracheostomy, or oropharyngeal tubes or other tubes or catheters) adapted to be intubated into one or more passageways (e.g., the trachea and/or pharynx) of a subject in connection with a medical procedure. For example, certain embodiments are directed toward endotracheal tubes inserted into a subject's trachea to facilitate mechanical ventilation of the subject's lungs. Certain embodiments include tubes having an improved configuration for periodic removal of fluids and/or solids that collect adjacent an inflatable cuff used to seal, secure, and/or position the tube against the tracheal wall. The inflatable cuff may include a partially inverted collar, which may be advantageous or desirable. For example, the partially inverted collar may reduce or eliminate possible occlusion of a suction opening. As used throughout this document, the term “subject” may include any human or other animal.

Turning to the drawings, FIG. 1 illustrates an example medical tube 10 including an inflatable cuff 12 having a partially inverted collar, in accordance with an example embodiment of the disclosure. Tube 10 may include a tubular body 16 having an open proximal end 18 and an open distal end 20. Tubular body 16 may define a gas-conveying passageway 22 for mechanical ventilation of a subject. Proximal end 18 may include a connector 24 configured for attachment to a mechanical ventilator (not shown).

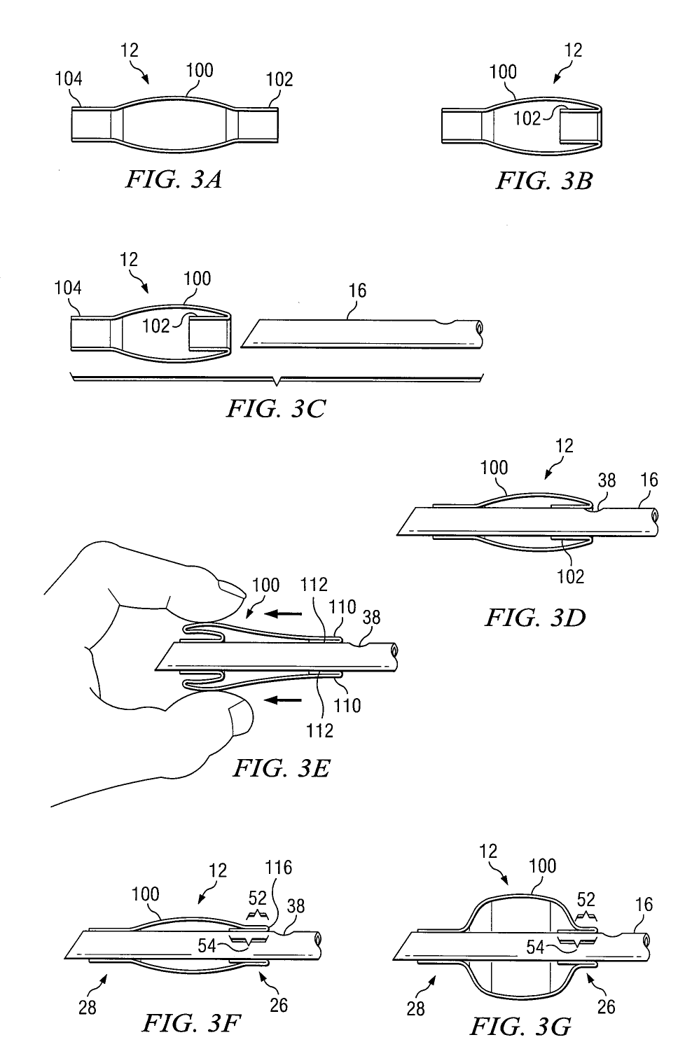

Inflatable cuff 12 may be mounted on tubular body 16, e.g., adjacent distal end 20 of tubular body 16. Cuff 12 may be mounted on tubular body 16 by one or more collars and/or any other suitable means. In the example embodiment shown in FIG. 1, cuff 12 may be mounted on tubular body 16 by a first collar 26 and a second collar 28. In this embodiment, first collar 26 is partially inverted such that a first portion of collar 26 is folded under a second portion of collar 26, as discussed below in greater detail with reference to FIGS. 2 and 3A-3G.

During intubation of tube 10, cuff 12 may be at least partially collapsed. Once properly in place, cuff 12 may be inflated via an inflation lumen 30 formed in or otherwise associated with tubular body 16. Inflation lumen 30 may be coupled to an inflation line 32 terminating in a fixture 34 that allows inflation of cuff 12 via inflation lumen 30.

Tubular body 16 may also include a suction lumen 36 formed in or otherwise associated with tubular body 16. Suction lumen 36 may include an opening 38 extending through the wall of tubular body 16 through which secretions or other matter accumulated on or proximate cuff 12 may be removed. In this embodiment, suction lumen 36 extends to the distal end 20 of tubular body 16 and includes a sealing plug 39. In other embodiments, suction lumen 36 may terminate before the distal end 20 of tubular body 16 (e.g., just beyond opening 38) or may terminate at opening 38. As shown, an exterior suction tube 40 may be communicatively coupled to lumen 36 for removing secretions or other matter through opening 38, as discussed below in greater detail. Suction tube 40 may include an end fixture 42 including a cap 44.

To insert and/or position tube 10 in the trachea, tube 10 may be inserted down the trachea to a point just above (e.g., about 3 cm above) the carina. Cuff 12 may then be inflated by pumping air into cuff 12 through inflation line 32 and inflation lumen 30. Typically, inflation air may be provided by a syringe inserted into fixture 34. In some situations, inflation of cuff 12 to a pressure of 25-30 cm H₂O (or any other clinically appropriate pressure level) may act to seal cuff 12 against the inner wall of the trachea, thus effectively sealing the trachea to prevent gas (e.g., gas pumped into the lungs through tube 10) from backing up around tube 10. Additionally, or alternatively, cuff 12 may act to secure or position tube 16 within the trachea.

Proximal end 18 of tubular body 16 may be attached to a ventilator using connector 24 for mechanical ventilation of the subject. Following intubation, fluid secretions and/or other matter may accumulate near the proximal end of cuff 12. These secretions may carry bacteria or other pathogens in an environment suitable for pathogen growth. Accordingly, the secretions may be periodically or continuously removed through opening 38, lumen 36, and suction tube 40. Cap 44 may be removed and fixture 42 may be connected to a suction machine (not shown) for removing the secretions; alternately, a syringe may be used for this purpose.

FIG. 2 is an enlarged view of cuff 12 shown in FIG. 1, in accordance with one embodiment of the disclosure. Cuff 12 is shown in its inflated state with collars 26 and 28 at respective ends of cuff 12 for attaching cuff 12 to tubular body 16. As discussed above, collar 26 may be partially inverted. For example, collar 26 may be folded back to form a partially inverted collar portion 50 that includes an outward collar portion 52 overlapping an inverted collar portion 54. Cuff 12 may be positioned so that suction opening 38 is located adjacent or proximate the fold of collar 26 to allow for the removal of secretions or other matter proximate cuff 12 via opening 38. In this embodiment, only collar 26 is folded or partially inverted; collar 28 is not folded or partially inverted. In other embodiments, both collars 26 and 28 may be folded or partially inverted, e.g., as collar 26 is shown in FIG. 2. In other embodiments, one collar may be partially inverted while the other collar is fully inverted.

As shown in FIG. 2, partially inverted collar 26 has a double thickness, which may help prevent opening 38 from contacting the tracheal wall and becoming occluded (e.g., during suctioning or otherwise), which occlusion may cause irritation and/or rubbing of the tracheal wall that may result in inflammation, scarring, and/or stenosis.

FIGS. 3A-3G illustrate an example method of configuring and attaching cuff 12 to tubular body 16 of medical tube 10, in accordance with one embodiment of the disclosure. FIG. 3A shows cuff 12, which can be manufactured from any suitable polymeric or other material, e.g., PVC (polyvinyl chloride), polyurethane, polyisoprene, and/or silicone. Cuff 12 may include an inflatable portion 100 and collar portions 102 and 104 at respective ends of cuff 12. As shown in FIG. 3B, collar portion 102 may be inverted such that collar portion 102 is disposed substantially within inflatable portion 100. Collar portion 102 may be inverted by reverse folding collar portion 102 at or proximate the junction or transition between collar portion 102 and inflatable portion 100.

As shown in FIG. 3C, cuff 12 with inverted collar portion 102 may be mounted on tubular body 16. Cuff 12 may be mounted on tubular body 16 in any suitable manner, e.g., by manually or automatically inserting an end of tubular body 16 through the opening defined by collar portions 102 and 104, or in any other suitable manner.

As shown in FIGS. 3D, cuff 12 may be mounted on tubular body 16 such that inverted collar portion 102 is located adjacent, partially covering, or completely covering suction opening 38 formed in tubular body 16. As shown in FIG. 3E, cuff 12 may be manipulated to configure collar portion 102 such that a first portion 110 of collar portion 102 is folded back over a second portion 112 of collar portion 102. For example, in the embodiment shown in FIG. 3E, inflatable portion 100 may be pulled back in the direction of the arrows in order to pull first portion 110 of collar portion 102 back over second portion 112.

Collar portion 102 may be manipulated in this (or similar) manner to form collar 26, as shown in FIG. 3F. As discussed above, collar 26 may include an outward collar portion 52 overlapping an inverted collar portion 54. Collar portions 52 and 54 may correspond to collar portions 110 and 112 shown in FIG. 3E.

The positioning of inverted collar portion 102 relative to opening 38 (FIG. 3D) and the amount that first portion 110 of collar portion 102 is pulled back over second portion 112 of collar portion 102 (FIG. 3E) may determine the position of the resulting collar 26 with respect to opening 38. In particular, such variables may determine the position of the folded transition between collar portions 52 and 54, indicated in FIG. 3F as fold 116, relative to opening 38. Thus, one or both of such variables may be controlled as desired in order to control the distance between fold 116 of collar 26 and opening 38 as desired. For example, in some embodiments, during the step shown in FIG. 3D, a portion of inverted collar portion 102 may be positioned over opening 38 such that when first portion 110 of collar portion 102 is pulled back over second portion 112 of collar portion 102 (as shown in FIG. 3E), the fold 116 of the resulting collar 26 may be located adjacent or immediately adjacent opening 38.

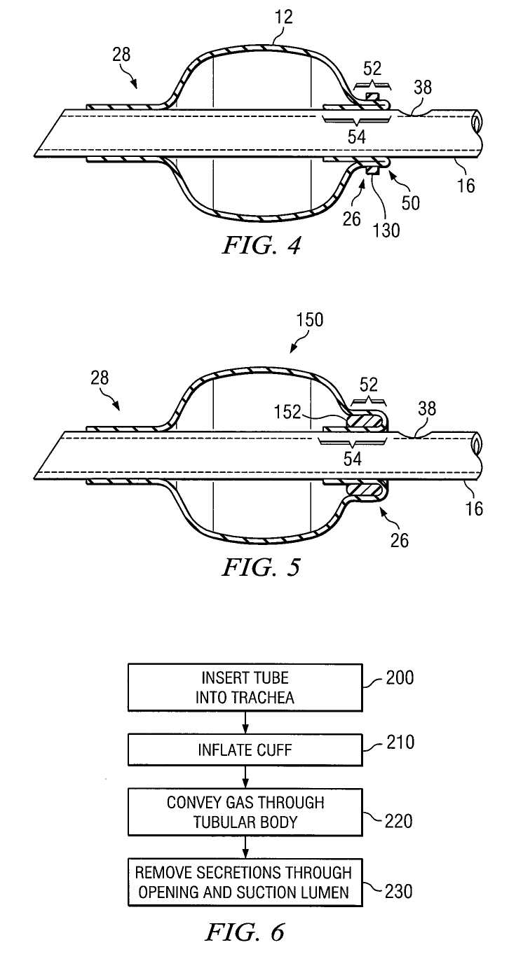

In some embodiments, collar portions 52 and 54 may be elastically stretched around tubular body 16, which may substantially secure collar 26 to tubular body 16. In some embodiments, all or portions of collars 26 and/or 28 may be bonded to tubular body 16 in any suitable manner. For example, in one embodiment, collars 26 and/or 28 may be bonded to an outer surface of tubular body 16 using a cyclohexanone solvent bond that may dissolve portions of collar 26/28 and tubular body 16 such that the surfaces of each become intermingled. As another example, a band or ring may be positioned around collars 26 and/or 28 to secure or help secure collars 26 and/or 28 to tubular body 16. An example of such band discussed below with respect to FIG. 4.

FIG. 3G shows cuff 12, having been mounted on tubular body 16 as shown in FIGS. 3A-3F, in an inflated state. In this example embodiment, collar 26 (including collar portions 52 and 54) and collar 28 may be secured to tubular body 16 such that collar 26 (including collar portions 52 and 54) and collar 28 do not inflate, while inflatable collar portion 100 inflates outwardly. Collars 26 and/or 28 may be secured to tubular body 16 in any suitable manner, e.g., by being elastically stretched around tubular body 16 and/or by being permanently secured by solvent bond, heat treatment (e.g., using RF, hot air, or ultrasonic techniques) or in any other manner.

In one embodiment, collar portions 102 and 104 (see FIG. 3A) of cuff 12 are about 0.012 inches thick. Thus, the double-thick, partially inverted collar 26 shown in FIGS. 3F and 3G may be about 0.025 inches thick.

As discussed above, in some embodiments or situations, this double-thick collar 26 may help reduce or eliminate possible occlusion of opening 38 that may prevent or reduce removal or secretions or other matter via opening 38. Further, in some embodiments or situations, by preventing contact of opening 38 with the tracheal wall, the double-thick collar 26 may help prevent irritation and rubbing of the tracheal wall that can result in inflammation, scarring, and/or stenosis. In addition, in some embodiments, tubular body 16 may include one or more projections located proximate opening 38, e.g., as described in co-pending PCT Application No. PCT/US2005/016577, filed May 11, 2005. Such projection(s) may further reduce or eliminate possible occlusion of opening 38.

FIG. 4 illustrates an example embodiment in which a band 130 is positioned around collar 26, according to one embodiment of the disclosure. Band 130 may be positioned around collar 26 to help secure collar 26 to tubular body 16 and/or to provide a thickened portion around collar 26, which may help prevent occlusion of opening 38 in tubular body 16.

Band 130 may have any suitable shape, size, and/or thickness, and may be located at any position along collar 26. Band 130 may be made from the same material as cuff 12 or from any other suitable material. Band 130 may be secured to or around collar 26 in any suitable manner. For example, band 130 may be an elastic band secured around collar 26 by elastic forces. As another example, band 130 may coupled to collar 26 using any suitable heat treatment (e.g., using RF, hot air, or ultrasonic techniques). As another example, band 130 may be secured to or bonded with collar 26 using a solvent.

In other embodiments, band 130 may instead be positioned around collar 28 to help secure collar 28 to tubular body 16. In other embodiments, bands 130 may be positioned around both collars 26 and 28.

FIG. 5 illustrates an example cuff 150 including a collar spacer 152, in accordance with one embodiment of the disclosure. Cuff 150 may be substantially similar or analogous to cuff 12 discuss herein, but may further include collar spacer 152 installed in partially-inverted collar 26 between collar portions 52 and 54, thus providing a thickened collar 26. Such thickened collar 26 may help reduce or eliminate possible occlusion of opening 38, which in some embodiments or situations, may provide one or more advantages discussed herein. In one embodiment, collar spacer 152 may be ring-shaped and may extend around the circumference of tubular body 16, sandwiched between collar portions 52 and 54. However, collar spacer 152 may have any suitable shape, configuration, and/or cross-section suitable to provide a thickened collar, at least proximate opening 38.

FIG. 6 is a flow diagram of a method of intubation of a tube having a cuff with a partially inverted collar, in accordance with one embodiment of the disclosure. At step 200, a tube may be inserted into a subject's trachea. The tube may have a tubular body and an inflatable cuff with a partially-inverted collar positioned proximate an opening in the tubular body for removing secretions or other matter via a suction lumen. The tube may have any suitable additional features, e.g., a Magill curve to facilitate intubation. At step 210, the inflatable cuff may be inflated to seal, secure, and/or position the tubular body against the tracheal wall. At step 220, gas may be conveyed (e.g., from a gas source) through the tubular body to an area of the subject's body (e.g., the lungs). At step 230, secretions or other matter proximate the inflatable cuff may be removed by suction through the opening in the tubular body and through the suction lumen.

The order of the steps discussed above can vary according to various embodiments. For example in some embodiments, gas may be conveyed through the tubular body prior to sealing/securing the tube to the tracheal wall using the inflatable cuff.

It will be appreciated that while the disclosure is particularly described in the context of endotracheal tubes, the apparatuses, techniques, and methods disclosed herein may be similarly applied in other contexts. For example, similar principles may be applied to a variety of other surgical and/or medical tubes having inflatable cuffs, e.g., tracheostomy tubes, oropharyngeal tubes, or other medical tubes or catheters. Additionally, it should be understood that various changes, substitutions and alterations can be made herein without departing from the spirit and scope of the disclosure as illustrated by the following claims.

What is claimed:

1. A medical device, comprising:
a tubular body configured to communicate gas; and
an inflatable cuff coupled to the tubular body at least by a collar;
wherein the collar includes a fold such that a first portion of the collar overlies a second portion of the collar in a concentric manner.

2. A medical device according to claim 1, further comprising a suction lumen extending along at least a portion of the tubular body and in fluid communication with a suction opening adjacent the collar.

3. A medical device according to claim 1, wherein the first portion of the collar lies directly on top of the second portion of the collar.

4. A medical device according to claim 1, further comprising a spacer located between the first portion and the second portion of the collar.

5. A medical device according to claim 4, wherein the spacer comprises a ring extending around the tubular body.

6. A medical device according to claim 1, wherein the first portion of the collar does not inflate upon inflation of the cuff.

7. A medical device according to claim 1, wherein:
the inflatable cuff further includes a second collar; and
the inflatable cuff is coupled to the tubular body at least by the two collars.

8. A medical device according to claim 7, wherein the second collar includes a fold such that a first portion of the second collar overlies a second portion of the second collar in a concentric manner.

9. A medical device according to claim 7, wherein the second collar does not include a fold.

10. A medical device according to claim 1, wherein the second portion of the first collar is permanently secured to the tubular body.

11. A medical device according to claim 1, wherein the medical device comprises an endotracheal tube.

12. A medical device according to claim 1, wherein the medical device comprises an tracheostomy tube.

13. A medical device according to claim 1, wherein the medical device comprises an oropharyngeal tube.

14. A medical device according to claim 1, wherein the medical device comprises a catheter.

15. A medical device, comprising:
conveying means for channeling gas to an area of the body;
securing means for securing the conveying means against the wall of a body cavity; and
attaching means for attaching the securing means to the conveying means, wherein the attaching means includes a fold such that a first portion of the attaching means overlies a second portion of the attaching means in a concentric manner.

16. A medical device according to claim 15, wherein the attaching means comprises a collar.

17. A method for intubation, comprising:
inserting at least a portion of a tube into a body cavity of a subject, wherein the tube comprises an inflatable cuff coupled to an tubular body by a collar, the collar including a fold such that a first portion of the collar overlies a second portion of the collar in a concentric manner;
inflating the cuff within the body cavity; and
conveying gas to an area of the subject's body through the tubular body.

18. A method according to claim 17, wherein inflating the cuff occurs prior to conveying gas through the tubular body.

19. A method according to claim 17, further comprising suctioning matter through an opening in the tubular body located proximate the collar of the inflatable cuff.

20. A method of attaching an inflatable cuff to a tubular body, the method comprising:
mounting an inflatable cuff on a tubular body, the inflatable cuff including a collar; and
manipulating the inflatable cuff to configure the collar such that a first portion of the collar overlies a second portion of the collar in a concentric manner.

21. A method according to claim 20, further comprising inverting the collar of the inflatable cuff prior to mounting the inflatable cuff on the tubular body.

22. A method according to claim 20, further comprising bonding the collar of the inflatable cuff to the tubular body.

23. A method according to claim 20, wherein:
the inflatable cuff further includes a second collar; and
manipulating the inflatable cuff comprises configuring the collar such that the first portion of the collar extends away from the second collar and the second portion of the collar extends toward the second collar.

24. A method according to claim 20, wherein a folded transition between the first and second portions of the collar is positioned adjacent a suction opening formed in the tubular body.

25. A method according to claim 20, further comprising positioning a spacer between the first and second portions of the collar.

26. A method according to claim 25, wherein the spacer is ring-shaped.

27. A method according to claim 20, further comprising permanently securing the second portion of the collar to the tubular body.

28. A method according to claim 20, wherein:
the inflatable cuff further includes a second collar; and
the inflatable cuff is mounted on the tubular body at least by the two collars.

29. A method according to claim 28, wherein the second collar includes a fold such that a first portion of the second collar overlies a second portion of the second collar in a concentric manner.

30. A method according to claim 28, wherein the second collar does not include a fold.
